(19) United States
(12) Patent Application Publication
(10) Pub. No.: US 2021/0353679 A1
(43) Pub. Date: Nov. 18, 2021

(54) PD-L1 PRESENTING PLATELETS REVERSE NEW-ONSET TYPE 1 DIABETES

(71) Applicant: NORTH CAROLINA STATE UNIVERSITY, Raleigh, NC (US)

(72) Inventors: Zhen GU, San Diego, CA (US); Jinqiang WANG, Raleigh, NC (US); Xudong ZHANG, Raleigh, NC (US)

(21) Appl. No.: 17/284,087

(22) PCT Filed: Oct. 10, 2019
(86) PCT No.: PCT/US2019/055524

§ 371 Date: Apr. 9, 2021

Related U.S. Application Data
(60) Provisional application No. 62/743,857, filed on Oct. 10, 2018.

(51) Int. Cl. A61K 35/19; A61P 3/10; A61K 47/69; C12N 5/078; C07K 14/705

(52) U.S. Cl. CPC A61K 35/19; A61P 3/10; C12N 2510/00; C12N 5/0644; C07K 14/70532; A61K 47/6901

(57) ABSTRACT

Disclosed are therapeutic agent delivery vehicle comprising a modified platelet comprising a therapeutic agent cargo and a targeting moiety and methods for treating diabetes, autoinflammatory disease, and/or graft vs host disease comprising administering the same to a subject.

This application claims the benefit of U.S. Provisional Application No. 62/743,857, filed on Oct. 10, 2018 which is incorporated herein by reference in its entirety.

### I. BACKGROUND

Type 1 diabetes (T1D) arises from breakdown of the immune regulation caused by genetic predisposition, environmental factors, and pathophysiology. Autoreactive lymphocytes destroy the insulin producing-β-cells, which leads the insufficient production of insulin and results in the uncontrolled blood glucose levels as well as many types of secondary complications. Infiltration of multiple types of lymphocytes has been detected in the pancreas of T1D patients. Among these pancreas-penetrating lymphocytes, the islet-antigen-reactive T cell plays a dominant role in the disease initiation and progression. These T cells can destroy the β-cells through T-cell receptor (TCR)-mediated cytotoxicity and production of cytokines, such as interferon-γ (IFN-γ). Due to the central role of the autoreactive lymphocytes in the pathogenesis of T1D, immune intervention holds great promise in treating T1D. T cells depletion with treatment of anti-CD3 monoclonal antibodies (teplizumab and otelixizumab) contributes to a sustained insulin production in the newly diagnosed patients. Although anti-CD3 antibody can reverse the new-onset T1D, however, this antigen non-specific intervention may cause adverse effects and safety concerns. Thus, what is needed are interventions of the islet antigen-specific T cell that can provide an enhanced safety to treat T1D with limited side effects.

### II. SUMMARY

Disclosed are methods and compositions related to engineered platelets comprising membrane bound PD-L1.

Disclosed herein are engineered platelets comprising membrane bound exogenous PD-L1. In one aspect, disclosed herein are engineered platelets of any preceding aspect further comprising membrane bound CD40L and/or toll-like receptors.

Also disclosed herein are engineered platelets of any preceding aspect further comprising a targeting moiety (such as, for example, a peptide, polypeptide, polymer, small molecule, nucleic acid, antibody, or sugar). It is understood and herein contemplated that the targeting moiety can be designed or engineered to target the bone marrow, liver, spleen, pancreas, prostate, bladder, heart, lung, brain, skin, kidneys, ovaries, testis, lymph nodes, small intestines, large intestines, or stomach.

In one aspect, disclosed herein are methods of treating/reducing/preventing/inhibiting diabetes, graft vs. host disease (GvHD), and/or an autoinflammatory disease or condition in a subject comprising administering to the subject the engineered platelets of any preceding aspect.

Also disclosed herein are methods of treating/reducing/preventing/inhibiting diabetes, graft vs. host disease (GvHD), and/or an autoinflammatory disease or condition of any preceding aspect, further comprising administering to the subject $-islet cells.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E 1F, 1G, 1H, 1I, 1J, 1K, and 1L show a schematic and production of PD-L1 presenting platelets. FIG. 1A shows a schematic of the production of PD-L1 platelets and inhibition of CD8⁺ T cells for β-cells protection. (I) Establishment of L8057 cell line stably expressing mouse PD-L1 and production of PD-L1 platelets. (II) PD-L1 platelets protect β-cells from autoreactive T cells via PD-1 blockade by PD-L1. FIG. 1B shows a confocal image of the L8057 cell line stably expressing mouse EGFP-PD-L1. WGA Alexa-Fluor 594 dye was used to stain cell membrane (Scale bar: 10 μm). c Analysis of the expression of PD-L1 on L8057 cell line by western blot. L8 is short for L8057 cells. FIGS. 1D and 1E show detection of CD41a in EGFP-PD-L1 L8057 cells by immunofluorescence staining and the flow cytometry (Scale bar: 10 μm). FIGS. 1F and 1G show detection of CD42a in EGFP-PD-L1 L8057 cells treated with 500 nM PMA by immunofluorescence staining and the flow cytometry (Scale bar: 10 μm). FIG. 1H shows different stages of PD-L1 MK cells that undergo maturation and differentiation (Scale bar: 10 μm). I: Mature EGFP-PD-L1 MK cells; II: Budding of proplatelets from MK cells; III: Extension of proplatelets from MK cells; IV: Release of proplatelets from MK cells. FIG. 1I show the morphology of PD-L1 proplatelets extended from L8057 cells (Scale bar: 10 μm). FIG. 1J shows confocal images of the purified PD-L1 platelets (Scale bar: 2 μm). FIG. 1K shows representative TEM image showing the morphology of PD-L1 platelets (Scale bar: 1 μm). FIG. 1L shows the size distribution of PD-L1 platelets measured by DLS.

FIGS. 2A, 2B, 2C, 2D, 2E, 2F, 2G, 2H, and 2I show in vitro and in vivo biological characterization of PD-L1 platelets. FIG. 2A shows representative TEM images of PD-L1 platelet, activated PD-L1 platelet and released platelet microparticles (PMPs). Scale bar in image I and II: 1 μm. Scale bar in image III: 100 nm. FIG. 2B shows measurement of the size distribution of PD-L1 platelets and PMPs at 30 min after activation by thrombin. FIG. 2C shows retention of PD-L1 platelets on the collagen-coated well for 30 min (Scale bar: 50 μm). FIG. 2D shows EGFP-PD-L1 platelets and free platelet bound on T cells (Scale bar: 10 μm). FIGS. 2E and 2F show representative plots (2e) and quantification (2f) of pancreas isolated GzmB⁺ CD8⁺ T cells of different treatment groups analyzed by the flow cytometry (Gated on CD8⁺ T cells) (n=5). Throughout, NS: no significant, *P<0.05, **P<0.01, ***P<0.001; two-way ANOVA with Tukey post-hoc test analyses were carried out to do the analyses. FIG. 2G show in vivo blood circulation retention property of free platelets and PD-L1 platelets. Fluorescence was measured at different time points as indicated (n=3). Error bar, ±s.d. FIG. 2H shows in vivo fluorescence images of biodistribution of free platelets and PD-L1 platelets in pancreas and the major organs. The mice were injected with NHS-Cy5.5 labeled free platelets and EGFP-PD-L1 platelets (200 μL, ˜2×10⁸), the distribution in organs was measured 20 h after the injection. FIG. 2I shows fluorescence intensity per gram of tissue in pancreas and the major organs as indicated (n=8). Error bar, ±s.d.

FIGS. 3A and 3B show that hPD-L1 platelets bind on human PD-1 positive T cells. Representative image (3a) and quantification (3b) of MEG-01 derived EGFP-PD-L1 platelets bound on CD3/CD28 Dynabeeds activated PD-1 positive T cells and unstimulated T cells (Scale bar: 10 μm).

FIG. 4 shows that CD8⁺ T cells were sorted viably for cell culture and expansion. Representative plots of CFSE⁺ CD8⁺ T cells of different treatment group analyzed by the flow cytometry (Gated on CD3⁺ T cells). The CD3⁺ T cells were incubated with PD-L1 platelets and Free platelets for 72 h, then were labeled with Carboxyfluorescein succinimidyl ester (CFSE) for 10 min, CD8⁺ T cells were then analyzed using a FACS with gated on CD3⁺ T cells.

FIGS. 5A, 5B, SC, 5D, 5E, and 5F show PD-L1 platelets reverse the hyperglycemia in the diabetic NOD mice. FIG. 5A shows blood glucose levels of the diabetic NOD mice with different treatments as indicated (n=12). FIG. 5B shows average blood glucose levels of diabetic NOD mice with different treatments as indicated (n=12). Dark green line: non-reversal diabetic NOD mice (n=3); Light green line: reversal diabetic NOD mice (n=9). Data represents as mean±s.d. FIGS. 5C and 5D show representative confocal images (5c) and quantify (5d) of insulin β-cells in the pancreas sections (Scale bar: 100 μm). FIG. 5E shows insulin level of the diabetic NOD mice after different treatments as indicated (n=12). (5C, 5G, 5I). FIG. 5F shows the occurrence of the NOD mice developed diabetes (n=10). Throughout, NS: no significant, *P<0.05, **P<0.01, ***P<0.001; one-way ANOVA with Tukey post-hoc test analyses were carried out to do the analyses (5d and 5e) or by Log-Rank (Mantel-Cox) test (5f).

FIGS. 6A and 6B show that PD-L1 platelets reverse the hyperglycemia in the diabetic NOD mice with 5 times treatment. FIG. 6A shows the treatment schedule. FIG. 6B shows blood glucose levels of the diabetic NOD mice with different treatments as indicated (n=12).

FIGS. 7A and 7B show that PD-L1 platelets reverse the hyperglycemia in the diabetic NOD mice with 10 times treatment. FIG. 7A shows the treatment schedule. FIG. 7B shows blood glucose levels of the diabetic NOD mice with different treatments as indicated (n=12).

FIGS. 8A, 8B, 8C, 8D, 8E, 8F, 8G, 8H, 8I, and 8J show characterizations of the T cell status in the pancreas of diabetic NOD mice receiving the platelets treatment. FIGS. 8A and 8B show representative confocal images (8a) and quantification (8b) of islet infiltrated CD8⁺ T cells by immunofluorescence staining (Scale bar: 100 μm). FIGS. 8C and 8D show representative plots (8c) and quantification (8d) of pancreas infiltrated CD3⁺ T cells in different treatment groups analyzed by the flow cytometry (Gated on CD3⁺ T cells) (n=12). FIGS. 8E and 8F show representative plots (8e) and quantification (8f) of pancreas-infiltrated CD8+ and CD4⁺ T cells in different treatment groups analyzed by the flow cytometry (Gated on CD3⁺ T cells) (n=12). FIGS. 8G and 8H show representative plots (8g) and quantification (8h) of pancreas infiltrated GzmB⁺ CD8⁺ T cells in different treatment groups analyzed by the flow cytometry (Gated on CD8⁺ T cells) (n=12). FIGS. 8I and 8J shows representative plots (8g) and quantification (8h) of pancreas infiltrated INF-γ⁺ CD8⁺ T cells in different treatment groups analyzed by the flow cytometry (Gated on CD8⁺ T cells) (n=12). Throughout, NS: no significant, *P<0.05, **P<0.01, ***P<0.001; one-way ANOVA with Tukey post-hoc test analyses were carried out to do the analyses (8b, 8d, 8f, 8h, and 8j).

FIGS. 9A and 9B show representative plots (9a) and quantification (9b) of FoxP3⁺ CD4⁺ T cells of the pancreas of different treatment group analyzed by the flow cytometry (Gated on CD8⁺ T cells) (n=12). Throughout, NS: no significant, *P<0.05, **P<0.01, ***P<0.001; one-way ANOVA with Tukey post-hoc test analyses were carried out to do the analyses.

FIGS. 10A and 10B show that the percentage of CD49b⁺ CD4⁺ Tr1 cells population in different treatment group of mice. Representative plots (10a) and quantification (10b) of CD49b⁺ CD4⁺ Tr1 cells of the pancreas of different treatment group analyzed by the flow cytometry (Gated on CD3⁺ T cells) (n=12). Throughout, NS: no significant, *P<0.05, **P<0.01, ***P<0.001; one-way ANOVA with Tukey post-hoc test analyses were carried out to do the analyses.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Controlled release” or “sustained release” refers to release of an agent from a given dosage form in a controlled fashion in order to achieve the desired pharmacokinetic profile in vivo. An aspect of “controlled release” agent delivery is the ability to manipulate the formulation and/or dosage form in order to establish the desired kinetics of agent release.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular PD-L1 expressing platelets are disclosed and discussed and a number of modifications that can be made to a number of molecules including the PD-L1 expressing platelets are discussed, specifically contemplated is each and every combination and permutation of PD-L1 expressing platelets and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

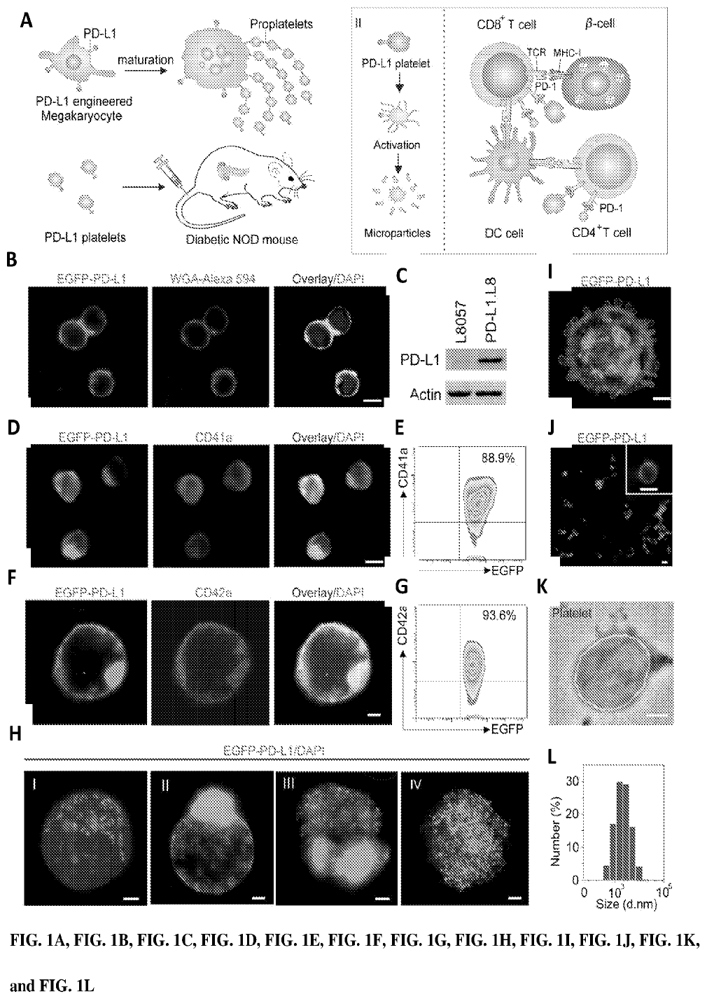

Self-antigen captured DCs play a crucial role on the peripheral tolerance through expanding CD4⁺ Foxp3⁺ Treg cells. Treg cells can directly restrain the activity of autoreactive T cells and NK cells to protect the β-cells from attack. To employ Treg cells to protect the β-cells, islet self-antigens (such as insulin B chain 9-23) have been developed to induce self-antigens specific Treg cells to treat T1D. Besides Treg cells, the normal tissues also express immune inhibitory ligands to inhibit the activity of the lymphocytes for maintaining peripheral tolerance. Programmed death-ligand 1 (PD-L1), a critical immune checkpoint ligand, presenting on the surface of normal tissue cells prevents autoimmune attack from CD8⁺ cytotoxicity T cells. The interaction of PD-L1 with programmed death-1 PD-1 (PD-1) receptor leads to T cell exhaustion. Deficient of PD-1/PD-L1 inhibitory axis leads to T1D in mice. Moreover, cancer patients receiving PD-1/PD-L1 blockade therapy have a risk to develop T1D, indicating that PD-L1 plays an important role in preventing the pathogenesis of T1D. Herein, platelets genetically presenting PD-L1 were utilized as an immunosuppressive modulator for restraining the activity of T cells and reversing the T1D diabetes in NOD mice (FIG. 1a). Accordingly, in one aspect, disclosed herein are engineered platelets comprising membrane bound exogenous PD-L1.

In addition to hemostasis and thrombosis, platelet also plays important functions in modulating inflammatory and immune response. For example, platelet contains potent immunoregulatory molecules, such as Toll-like receptors (TLRs) and CD40L, which can directly interact with innate immune cells including T cells, DC cells, and neutrophils. Thus, in one aspect, disclosed herein are engineered platelets of expressing membrane bound PD-L1, further comprising membrane bound CD40L and/or toll-like receptors.

Platelets can also bind and inhibit the activity of T lymphocyte and contributes to anti-inflammatory therapy in rheumatoid arthritis. In addition, platelets also contain multiple anti-inflammatory cytokines including transforming growth factor β (TGF-β), which can inhibit T cell function, dampening host's cancer immunity. In this study, it was demonstrated that a combination of the physiological properties and incorporated immune blockade function of the engineered platelets can be leveraged to reverse the new-onset T1D in an NOD mouse model.

It is understood and herein contemplated that eh disclosed engineered platelets expressing membrane bound PD-L1 are designed to target the PD-L1 to T cells infiltrating a particular tissue or organ site. One way to direct the platelets to a particular tissue or organ site of interest is through the use of a targeting moiety. For example, the targeting moiety can be designed to or engineered to target the bone marrow, liver, spleen, pancreas, prostate, bladder, heart, lung, brain, skin, kidneys, ovaries, testis, lymph nodes, small intestines, large intestines, or stomach. It is understood and herein contemplated that there are a number of approaches that can target the engineered platelets disclosed herein to a target tissue or organ. Thus, specifically contemplated herein are engineered platelets comprising any molecule that can be linked to the modified platelet for targeting a specific tissue or organ including, but not limited to peptides, polypeptides, polymers, nucleic acids, antibodies, sugars, or cells. In one aspect, the platelet is chemically conjugated to the targeting moiety.

It is understood and herein contemplated that engineered platelet can be linked to the targeting moiety through a chemical linkage or conjugation. In one aspect, disclosed herein are engineered platelets expressing membrane bound PD-L1, wherein the platelet is chemically conjugated to the targeting moiety via copper(I) catalyzed [3+2] azide-alkyne cycloaddition (CuAAC), strain-promoted azide-alkyne cycloaddition (SPAAC), Strain-promoted alkyne-nitrone cycloaddition (SPANC), or Dibenzocyclooctyl (DBCO) Copper-Free cycloaddition (for example, a Dibenzocyclooctyl (DBCO)-polyethylene glycol (PEG) 4 NHS ester). To facilitate the conjugation, the targeting moiety can also be modified to complete the linkage to the platelet. Accordingly, disclosed herein are therapeutic agent delivery vehicles of any preceding aspect, wherein the targeting moiety is treated with an activated azide molecule (such as, for example, N-azidoacetylgalactosainine-tetraacylated (Ac4GalNAz)).

### 1. Pharmaceutical Carriers/Delivery of Pharmaceutical Products

In one aspect, it is understood that the therapeutic agent delivery vehicles disclosed herein are intended for administration to a subject to treat, prevent, inhibit, or reduce diabetes, graft vs. host disease (GvHD), and/or an autoinflammatory disease or condition. Thus, disclosed herein are pharmaceutical compositions comprising any of the engineered platelets disclosed herein.

In one aspect, disclosed herein are pharmaceutical compositions comprising any engineered platelet expressing membrane bound PD-L1 disclosed herein and a targeting moiety; wherein the platelet has been modified to comprise a therapeutic agent cargo and a chemical linkage; wherein the chemical linkage comprises Dibenzocyclooctyl (DBCO)-polyethylene glycol (PEG) 4 NHS ester; and wherein the platelet is chemically conjugated to the targeting moiety; wherein the one or more therapeutic cargo agents comprise, a small molecule (including, but not limited to 1-methyl-tryptophan (1-MT), norharmane, rosmarinic acid, epacadostat, navooximod, doxorubicin, tamoxifen, paclitaxel, vinblastine, cyclophosphamide, and 5-fluorouracil), siRNA, peptide, polymer, peptide mimetic, and/or antibody (such as, for example, and anti-PDL-1 antibody including, but not limited to nivolumab, pembrolizumab, pidilizumab, BMS-936559, Atexolizumab, Durvalumab, and Avelumab).

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. METHODS OF TREATING TYPE 1 DIABETES, GRAFT VS HOST DISEASE, AND/OR AUTOINFLAMMATORY DISEASE OR CONDITIONS

As noted herein, the disclosed engineered platelets and/or pharmaceutical compositions can be used to treat, prevent, inhibit, or reduce diabetes, graft vs. host disease (GvHD), and/or an autoinflammatory disease or condition. Accordingly, disclosed herein are methods of treating, preventing, inhibiting, or reducing diabetes, graft vs. host disease (GvHD), and/or an autoinflammatory disease or condition in a subject the disclosed engineered platelets expressing membrane bound PD-L1 and/or pharmaceutical compositions. In one aspect, the methods can platelets used in the disclosed methods can further express membrane bound CD40L and/or toll-like receptors.

It is understood and herein contemplated that the autoinflammatory disease or condition that can be treated, inhibited, prevented, or reduced through the administration of the engineered platelets disclosed herein include, but are not limited to Achalasia, Acute disseminated encephalomyelitis, Acute motor axonal neuropathy, Addison's disease, Adiposis dolorosa, Adult Still's disease, Agammaglobulinemia, Alopecia areata, Alzheimer's disease, Amyloidosis, Ankylosing spondylitis, Anti-GBM/Anti-TBM nephritis, Antiphospholipid syndrome, Aplastic anemia, Autoimmune angioedema, Autoimmune dysautonomia, Autoimmune encephalomyelitis, Autoimmune enteropathy, Autoimmune hemolytic anemia, Autoimmune hepatitis, Autoimmune inner ear disease (AIED), Autoimmune myocarditis, Autoimmune oophoritis, Autoimmune orchitis, Autoimmune pancreatitis, Autoimmune polyendocrine syndrome, Autoimmune retinopathy, Autoimmune urticaria, Axonal & neuronal neuropathy (AMAN), Bald disease, Behcet's disease, Benign mucosal emphigoid, Bickerstaffs encephalitis, Bullous pemphigoid, Castleman disease (CD), Celiac disease, Chagas disease, Chronic fatigue syndrome, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diabetes mellitus type 1, Discoid lupus, Dressler's syndrome, Endometriosis, Enthesitis, Eosinophilic esophagitis (EoE), Eosinophilic fasciitis, Erythema nodosum, Essential mixed cryoglobulinemia, Evans syndrome, Felty syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Inflamatory Bowel Disease (IBD), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus nephritis, Lupus vasculitis, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjögren's syndrome, Sperm & testicular autoimmunity, Stiff person syndrome (SPS), Subacute bacterial endocarditis (SBE), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Urticaria, Urticarial vasculitis, Uveitis, Vasculitis, Vitiligo, Vogt-Koyanagi-Harada Disease, and Wegener's granulomatosis (or Granulomatosis with Polyangiitis (GPA).

In one aspect, the disclosed methods of treating/reducing/preventing/inhibiting diabetes, graft vs. host disease (GvHD) (such as, for example, GvHD of transplanted $-islet cells or kidneys), and/or an autoinflammatory disease in a subject comprising administering to the subject any of the engineered platelets cells expressing membrane bound PD-L1 disclosed herein can comprise administration of the engineered platelets at any frequency appropriate for the treatment, reduction, prevention, and/or inhibition of diabetes, graft vs. host disease (GvHD), and/or an autoinflammatory disease. For example, the engineered platelets can be administered to the patient at least once every 12, 14, 16, 18, 20, 22, 24, 26, 28, 30, 32, 34, 36, 38, 40, 42, 44, 46, 48 hours, once every 3, 4, 5, 6, 7, 8, 9, 10, 11, 12, 13, 14, 15, 16, 17, 18, 19, 20, 21, 22, 23, 24, 25, 26, 27, 28, 29, 30, 31 days, once every 2, 3, 4, 5, 6, 7, 8, 9, 10, 11, or 12 months. In one aspect, the engineered platelets is administered at least 1, 2, 3, 4, 5, 6, 7 times per week.

In one aspect, it is understood and herein contemplated that the methods of treating/reducing/preventing/inhibiting diabetes, graft vs. host disease (GvHD), and/or an autoinflammatory disease or condition can further comprise administering to the subject β-islet cells. β-islet cells can be administered before, concurrent with, simultaneously with, or following administration of the engineered platelets. In one aspect, the engineered platelets are administered at least 1, 2, 3, 4, 5, 6, 7, 8, 9, 10, 11, 12, 13, 14, 15, 16, 17, 18, 19, 20, 21, 22, 23, 24, 30, 36, 42, 48 hours, 3, 4, 5, 6, 7, 8, 9, 10, 11, 12, 13, 14 days, 3, 4, 5, 6, 7, 8 weeks prior to the administration of the $-islet cells.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

a) Results

(1) Establish Megakaryocytes (MKs) Cell Line Stably Expressing PD-L1.

Platelets are originally released into the blood from the mature MKs resident in the bone marrow. To produce platelets in vitro, murine MKs progenitor cell line L8057 was employed. L8057 cells underwent the process of the maturation, differentiation and platelet release after stimulated with phorbol 12-myristate 13-acetate (PMA). To genetically engineer the L8057 cell stably expressing PD-L1, L8057 cells were infected with the lenti-virus encoding murine PD-L1. Subsequently, the infected cells were selected with puromycin to obtain the stable cell line. As indicated by the cell membrane dye Alexa Fluor 594 conjugate wheat germ agglutinin (WGA594), EGFP-PD-L1 was overexpressed and localized on the cell membrane of the L8057 cells (FIG. 1b). The expression of EGFP-PD-L1 was further examined by western blot in L8057 cells (FIG. 1c). Furthermore, the MK cells marker CD41a was detected on the EGFP-PD-L1 L8057 cells (FIGS. 1d and 1e). CD42, the marker indicating maturation of MKs, intensively expressed in L8057 cells with the stimulation of PMA (FIGS. 1f and 1g). Additionally, the platelet markers including GPVI and P-selectin were also detectable in the mature PD-L1 L8057 cells.

With the stimulation of PMA, the PD-L1 positive vesicles were accumulated in the plasma of the mature L8057 cells (FIGS. 1h and 1i). Subsequently, the proplatelets budded and extended from the cell membrane (FIGS. 1h, and 1i). Finally, the fragmentation of the proplatelets released the platelets (FIG. 1h). The platelets presenting EGFP-PD-L1 were collected and purified from the culture medium (FIG. 1j). The isolated PD-L1 presenting platelet showed as spherical morphology under the transmission electron microscopy (TEM) (FIG. 1k). The dynamic light scattering (DLS) analysis demonstrated that the average diameter of the PD-L1 platelets was around 1.5 μm and with a zeta potential of ˜−10 mV (FIG. 1l). After stimulating with thrombin, the expression of P-Selectin was detected on the activated platelets. Phosphatidylserine was also presented on the surface of the activated platelets, indicating that the platelets underwent death after activation.

(2) Biological Characteristics of PD-L1 Platelets.

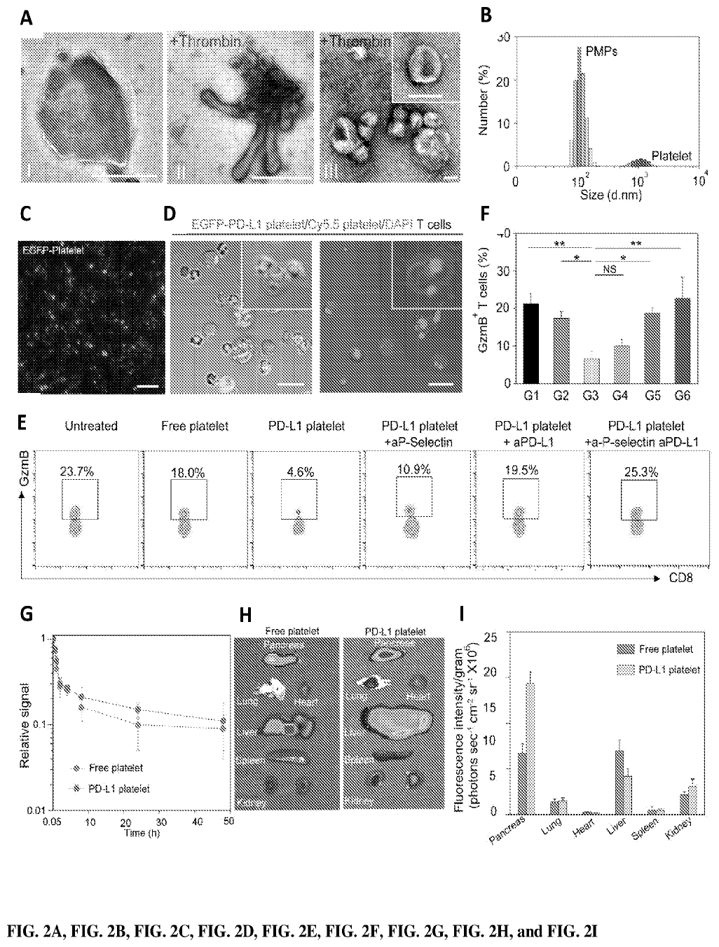

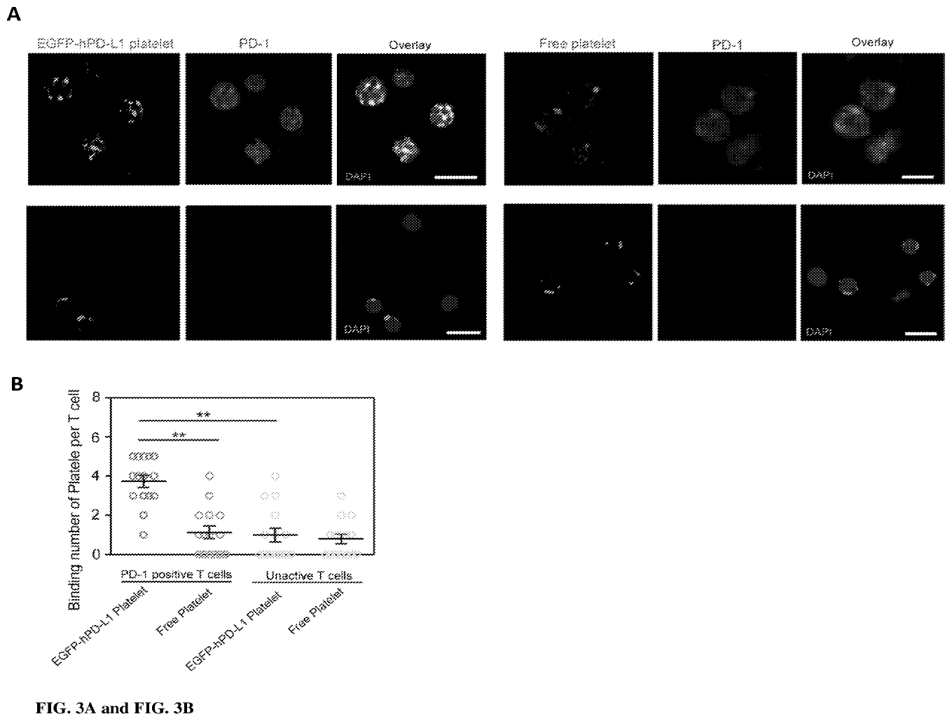

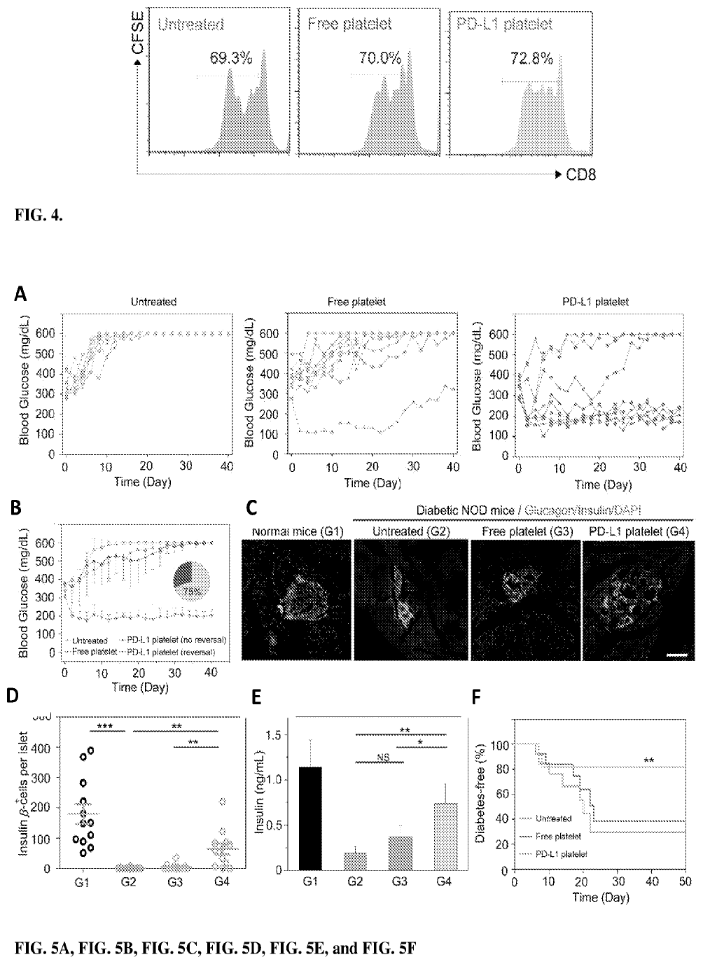

Platelet microparticles (PMPs) are fragments shed from the activated platelets, which also play the function of platelets in hemostasis, thrombosis, inflammation and promoter of tissue regeneration. To examine whether PMPs can be generated from the activated PD-1-expressing platelets, the platelets were treated with thrombin in vitro. After stimulation with thrombin, the engineered platelets were activated and showed an amorphous form with multiple tentacles (FIG. 2a). The TEM images also showed the generation of PMPs from activated platelets with an average diameter of ˜100 nm (FIGS. 2a and 2b). The number of blood circulated PMPs increases in several prothrombotic and inflammatory disorders, and some cancers. To investigate whether PD-L1 platelets can release the PMPs in NOD mouse with was observed to release from the platelets in vivo. Most of the platelets were individual cells, indicating the low thrombosis potential of the PD-L1 platelets. PMPs have a significantly smaller size compared to resting platelets, which enhances pancreas infiltration of PD-L1 presenting particles and further interaction with T cells. Rupture of a blood vessel leads to the exposure of collagen protein, which can recruit the platelets to execute hemostasis. To test the function of collagen binding effect of the PD-L1 platelets, PD-L1 platelets were incubated with the collagen coated well in vitro. Of note, EGFP-PD-L1 platelets can effectively adhere to collagen-coated wells (FIG. 2c). On the other hand, thrombus formation is another critical event for hemostatic response. After activation by thrombin, PD-L1 platelets bound with each other and formed the aggregation. Next, the interaction between PD-L1 platelets and the T cells in vitro was detected. The CD90.2⁺ T cells pancreas isolated from pancreas of the 16 weeks of the NOD mice with hyperglycemia (blood glucose >500 mg/dL) were incubated with PD-L1 platelets and free platelets, respectively. Both of PD-L1 platelets and free platelets can bind with T cells (FIG. 2d). Importantly, the frequency of GzmB positive CD8⁺ T cells were significantly decreased after incubated with PD-L1, indicating that PD-L1 platelets can exhaust CD8⁺ T cells (FIGS. 2e and 2f). Moreover, the free platelets had a significantly lower effect on the activity of CD8⁺ T cells (FIGS. 2e and 2f). This limited suppressive effect has been reported to be P-Selectin dependent. In addition, platelet derived TGF-β also dampen the host's immune response. The TGF-β1 from the culture medium and released from the platelets was also detected, which contributes to the therapy of T1D. Furthermore, the human megakaryocyte cell line MEG-01 was genetically engineered and stably expressed human PD-L1 (hPD-L1) and underwent maturation and differentiation. Similarly, hPD-L1 platelets were able to bind on the human PD-1 positive T cells and restrain their activity, and have limited effect on the vitality and proliferation (FIG. 3A, 3B, and FIG. 4)

To investigate the systemic circulation of the engineered platelets, the PD-L1 platelets were labeled with Cy5.5 and subsequently injected into NOD mice with hyperglycemia through tail-vein. The PD-L1 platelets showed a similar blood retention as the free platelets (FIG. 2g) and the half-life (t 1/2) of the PD-L1 platelets and free platelets was around 30.6 h and 23.9 h, respectively. Next, the in vivo tissue biodistribution of the PD-L1 platelets was investigated in NOD mice with hyperglycemia. Notably, the promoted EGFP-PD-L1 platelets and free platelet were able to accumulate in the pancreas of NOD mice (FIGS. 2h and 2i). with high glucose levels can be observed compared to the NOD mice treated with free platelets (FIGS. 2h and 2i). Moreover, the PD-L1 platelets also were shown priority to accumulate in the pancreas of the diabetic NOD mice compare to that of the healthy mice. Meanwhile, PD-L1 platelets also accumulated in the liver intensively (FIGS. 2h and 2i).

(3) PD-L1 Platelets Reverse the New-Onset T1D in NOD Mice.

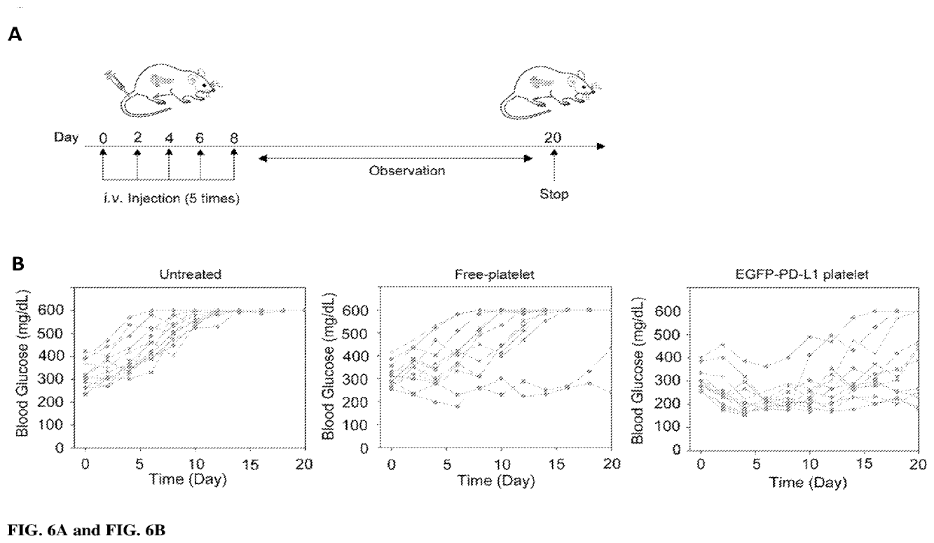

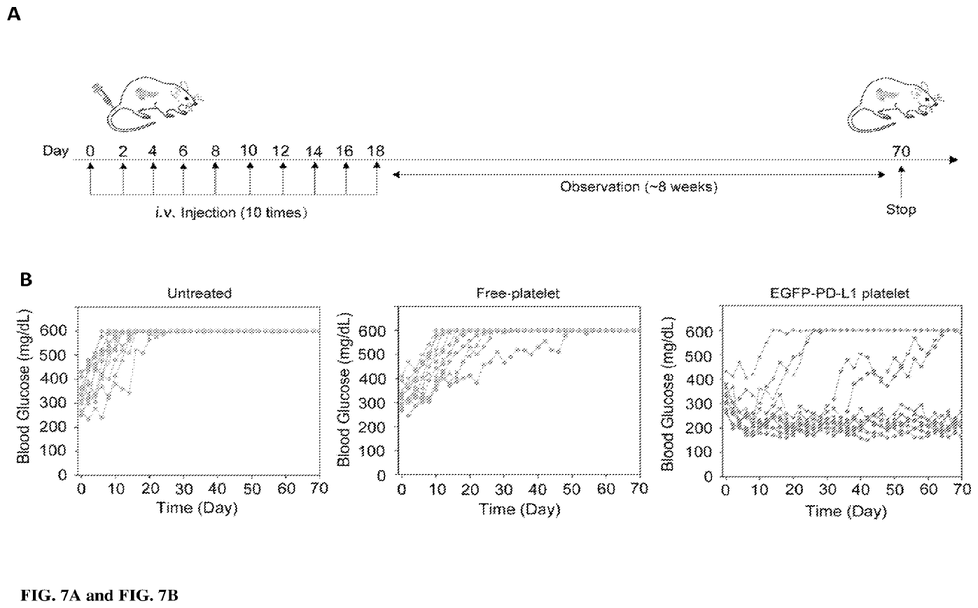

PD-L1 plays a crucial role in maintaining the peripheral immune tolerance, which contributes to controlling the activity of T cells. Thus, the PD-L1 presenting platelets were supposed to function as immunosuppressive cells to protect the β-cells from the attack of islet-specific autoreactive T cells. To investigate whether PD-L1 platelets can reverse the new-onset T1D, the NOD mice were divided into three groups, and the blood glucose was tested every two days at 10 weeks of age. Healthy maintained normoglycemia with the blood glucose from 80 to 130 mg/dL. Once the blood glucose level of the NOD mice was over 250 mg/dL, the mice were considered to exhibit new-onset diabetes. Then, the diabetic NOD mice were intravenously injected with either the free platelets or PD-L1 platelets every two days until endpoint (40 days), respectively. As shown in FIG. 5a, when the new-onset T1D in NOD mice (blood glucose >250 mg/dL) were left untreated, the blood glucose was increased gradually and finally reached hyperglycemia (blood glucose >600 mg/dL). In contrast, for the new-onset T1D mice received the treatment of PD-L1 platelets, the progress of new-onset T1D of were remarkably inhibited in 75% mice and the hyperglycemia were reversed to normoglycemia (9 of 12 total mice) (FIGS. 5a and 5b). However, treatment of the new-onset T1D mice with the free platelet, had limited effect on the inhibition of the progress T1D, and could not reverse hyperglycemia (FIGS. 5a and 5b). To further examine the insulin production β-cells, the pancreas of the NOD mice from different treatment groups were collected and analyzed by immunofluorescence. As shown in FIG. 5c, the insulin production β-cells were intact in the NOD mice with normoglycemia (blood glucose <130 mg/dL). In contrast, most of the β-cells were lost in the NOD mice with hyperglycemia (blood glucose >500 mg/dL) (FIGS. 5c and 5d). Of note, NOD mice with the treatment of PD-L1 platelets partially prevented the damage and loss of the insulin production β-cells (FIGS. 5c and 5d). Conversely, NOD mice treated with free platelets could not prevent the loss of the β-cells (FIGS. 5c and 5d). Furthermore, the level of the blood insulin of the NOD mice was also examined. With the treatment with PD-L1 platelets, the insulin levels were increased by 3-fold compared with the untreated NOD mice (FIG. 5e). In order to check short-term therapeutic effect of PD-L1 platelets, the diabetic NOD mice were treated with control platelets and PD-L1 platelets 5 times (10 days) and 10 times (20 days), respectively. It was observed that diabetic NOD mice who received 5 times treatments maintained normoglycemia during the treatment period, however, only 41% of the mice still maintains normoglycemia at day 20 (FIGS. 6A and 6B). Diabetic NOD mice which received 10 times treatments (20 days) with PD-L1 platelets achieved similar benefit compared to the mice which received 20 treatments (FIG. 5a). Most of the mice (75%) maintained normoglycemia at day 30 (FIGS. 7A and 7B). To investigate whether the mice could achieve long-term benefits after the PD-L1 platelets treatment, the blood glucose level of the mice who received 10 PD-L1 platelet treatments after day 20 was measured. During the following 8 weeks, 58% of the PD-L1 platelet treated mice reversed to normoglycemia (7 of 12 total mice). This data indicated that the mice could achieve long-term benefits after the PD-L1 platelets treatment. To investigate the effect of PD-L1 on preventing the diabetes in NOD mice, the NOD mice were treated with normoglycemia at 10 weeks of age. Strikingly, PD-L1 platelets treatment resulted in a significant reduction in diabetes incidence in the diabetic NOD mouse model compared with the NOD mice treated with free-platelets (P<0.01, Kaplan-Meier estimate) (FIG. 5f).

(4) PD-L1 Platelets Exhaust Pancreas-Penetrated T Cells.

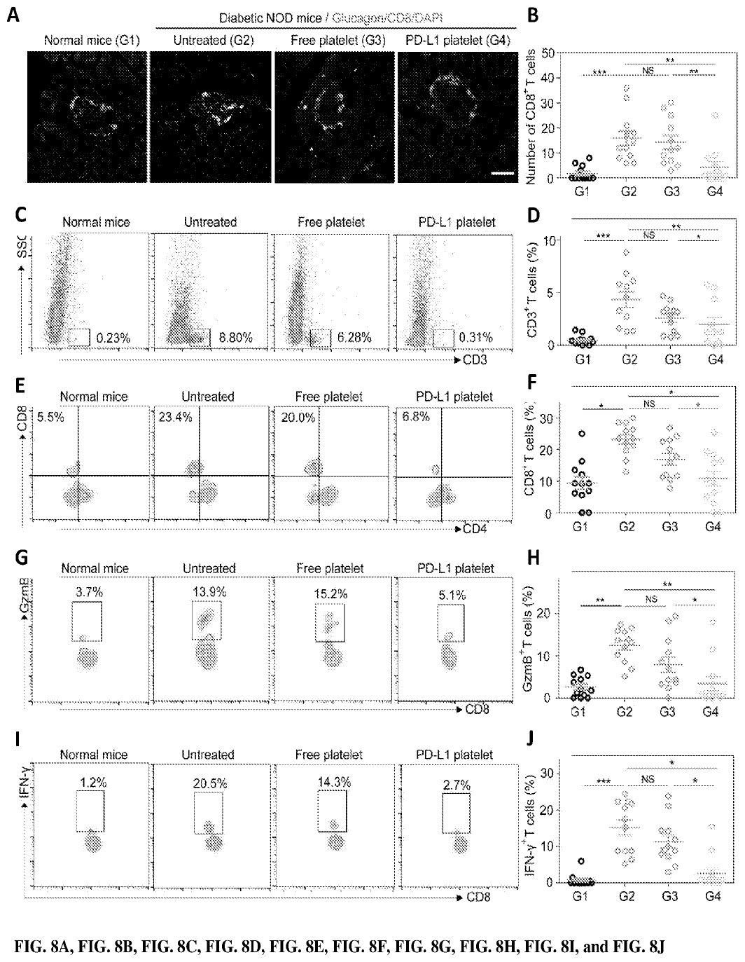

Pancreas-infiltrated autoreactive T cells attack β-cells cause T1D. To examine the status of pancreas-infiltrated T cells, the pancreas of the NOD mice from different treatment groups was collected and analyzed by immunofluorescence. As shown in FIG. 8a, there were few CD3+ or CD8⁺ T cells penetrating the pancreas in the NOD mice with normoglycemia, but intensive T cells penetrating the pancreas margin and islets in the NOD mice with hyperglycemia (FIGS. 8a and 8b). With the treatment of PD-L1 platelets, the pancreas-penetrated CD8⁺ T cells were significantly reduced (FIGS. 8a and 8b). In contrast, the free platelets had a limited effect on preventing T cell penetration (FIGS. 8a and 8b). The pancreas-penetrated T cells were further analyzed by flow cytometer. CD3⁺ T cell frequency was significantly increased in the hyperglycemia NOD mice compared to that associated with the normoglycemia NOD mice (FIGS. 8c and 8b). Strikingly, treatment of PD-L1 platelets intensively inhibited pancreas T cell penetration compared to the mice treated with free platelets (FIGS. 8c and 8d). Moreover, the frequency of CD8⁺ T cells was significantly reduced in the pancreas of the NOD mice treated with PD-L1 platelets compared to that of untreated hyperglycemia NOD mice (FIGS. 8e and 8f); while the diabetic NOD mice with treatment of free platelet had a limited effect on the frequency of CD8⁺ T cell penetration (FIGS. 8e and 8f). Activated CD8⁺ toxicity T cells secrete immune cytokines including interferon gamma (IFN-γ), granzyme B and perforin to attack the β-cells. As displayed in FIGS. 8g, 8h, 8i, and 8j, in these untreated hyperglycemia NOD mice, the pancreas-penetrated CD8⁺ T cells were GzmB and IFN-γ positive, indicating that T cells can cause the damage of the β-cells. Of note, PD-L1 platelets inhibit the activity of the CD8⁺ T cells compared to the NOD mice that received the free platelet treatment (FIGS. 8g, 8h, 8i, and 8j).

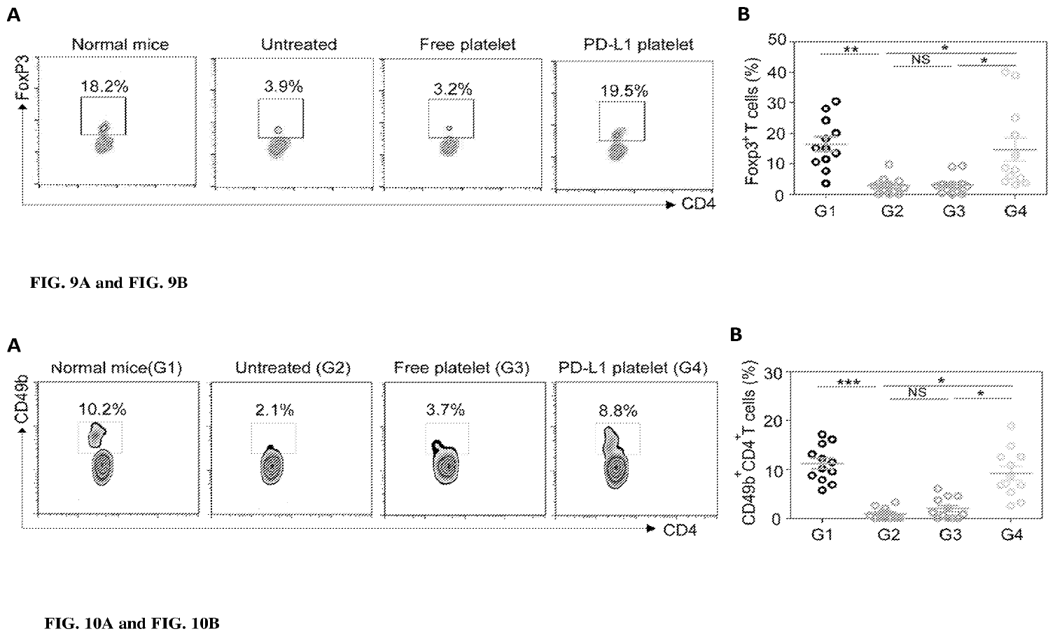

The CD4+CD25+FoxP3+ Tregs cells function as suppressor T cells, maintaining tolerance to self-antigens, and preventing autoimmune disease including T1D. The flow cytometer results revealed that the frequency of Tregs was significantly reduced in the untreated hyperglycemia NOD mice (FIG. 9a). Under the treatment of PD-L1 platelet, the loss of Tregs had also been prevented, which can devote to the β-cells protection (FIG. 9b). Another type of regulatory T cell, the CD49b⁺ CD4⁺ regulatory T (Tr1) cell, also plays a critical role in repressing immunity in autoimmune disease. Nanoparticles coated with major histocompatibility complex class II (pMHCII) molecules present self-antigen to trigger expansion of Tr1, contributing to the treatment of autoimmune disease including T1D. Here it was also observed that Tr1 cells were restored in the pancreas of the mice received the treatment of PD-L1 platelets (FIGS. 10A and 10B). Collectively, it was demonstrated that the PD-L1 platelets can effectively inhibit the activity of pancreas-penetrated CD8⁺ T cells and increased the percentage of the Tregs, which contributed to reverse the new-onset T1D in the NOD diabetic mice.

b) Discussion

In summary, infusion of PD-L1 platelets could inhibit the progress and reverse the new-onset type 1 diabetes in NOD mice. PD-L1 presenting platelets and its released PMPs accumulated in the inflamed pancreas and execute the immunosuppressive function. The activity of the pancreas penetrated effect T cells had been intensively inhibited and the insulin producing β-cells were rescued, leading to the reversal of hyperglycemia to normoglycemia. Furthermore, PD-L1 platelet treatment also increased the percentage of the Tregs in the pancreas and enhanced the pancreas immune tolerance, which also contributed to the reversal of the new-onset T1D in the NOD mice. This immune checkpoint blockaded-mediated cell therapy strategy can be further extended to treat other autoimmune diseases with targeting capability and limited side effects.

c) Methods

(1) Chemical and Regents.

Thrombin and anti-mouse PD-L1 antibody were purchased from Sigma-Aldrich. Anti-mouse CD4, CD8, CD41a and CD42a antibodies used for immunofluorescent staining were purchased from Abcam. Mouse GPVI antibody was purchased from R&D Systems (MAB6758). P-Selection (sc-8419) antibody was purchased from Santa Cruz biotechnology. The antibodies (Anti-CD41a, CD42d, CD3, CD4, CD8, Foxp3, GrzmB and IFN-γ) used for fluorescence-activated cell sorting (FACS) were purchased from Biolegend Inc. Wheat Germ Agglutinin (WGA) Alexa 594 dyes was purchased from Thermo Scientific.

(2) Cell Culture.

L8057 cells were cultured in Roswell Park Memorial Institute (RPMI) (RPMI) 1640 medium supplemented with 20% Fetal Bovine Serum (FBS). HEK293T cells were cultured in Dulbecco's Modified Eagle's Medium (DMEM) supplemented with 10% FBS.

(3) Establish Stable Cell Line.

Lenti vector encoding murine PD-L1 and human PD-L1 with C-terminal monomeric GFP tag (pLenti-C-mGFP-PD-L1-puro) and the packaging plasmids were purchased from Origene Technology. HEK293T cells were transiently transfected with the PD-L1 plasmids and the packaging plasmids according to the manufacturer's instructions. 48 h after the transfection, lenti-virus was iaosalted and purified from the culture medium. Then, L8057 cells were infected with the lenti-virus and incubated with 6 μg/ml polybrene. After infecting for 96 h, L8057 cells were incubated with 1 μg/mL puromycin to screen the cell line stable expressing mouse PD-L1. The established EGFP-PD-L1 expressing L8057 cells were maintained in 20% FBS complementary with 0.5-1 μg/ml puromycin.

(4) Production of Platelet.

EGFP-PD-L1 stably expressing L8057 cells were cultured in 1640 medium supplemented with 500 nM PMA for 3 days. After that, the mature L8057 cells were cultured for another 6 days for differentiation. The platelets were released into the culture medium after the differentiation. The culture medium was collected to isolate the platelets. The culture medium was firstly centrifuged at 1000 rpm for 20 min to remove the L8057 cells. Subsequently, the supernatant was centrifugation at 12,000 rpm for 30 min. The platelet precipitate was finally resuspended carefully in PBS with 1 μM PGE1 or Tyrode's buffer (134 mM NaCl, 12 mM NaHCO₃, 2.9 mM KCl, 0.34 mM Na₂HPO₄, 1 mM MgCl₂, 10 mM HEPES, pH 7.4).

(5) Immunofluorescent Assay.

L8057 cells were fixed with 4% paraformaldehyde for 10 mins. Then, the cells were washed with PBS for three times. Then, the fixed cells were incubated with 3% BSA and 0.2% Triton X-100 for blocking and permeabilization. After that, L8057 cells were incubated with primary antibodies as indicated overnight at 4° C., respectively. At the second day, the cells were washed with PBS for three times to remove the unbound antibodies. Subsequently, the cells were incubated with rhodamine-conjugated secondary antibody (1.5% BSA) 1 h in dark. The nucleus was then stained with DAPI for 20 mins. Finally, the cells were washed with PBS three times. The cells were observed by confocal microscopy (Zeiss) using a 40× objective.

(6) Western Blot Assay.

Western blot was performed as described. Briefly, EGFP-PD-L1 L8057 cells were lysed with loading buffer. The samples were boiling water baths for 15 mins. Subsequently, the samples were subjected in 12% SDS-PAGE. The proteins were transferred to the PVDF membrane and analyzed using PD-L1 and β-actin primary antibodies.

(7) In Vitro T Cell Binding and Activity Assay.

Pan T cells (CD90.2+ T cells) were isolated from the pancreas of the NOD mice using a T cell isolation kit (Thermo Fisher). EGFP-PD-L1 platelets (˜1×10⁸) or Cy5.5 labeled free platelets (˜1×10⁸) were incubated with the T cells overnight. After that, the nucleus was stained with Hoechst for 10 min. The binding of the platelets and T cells was observed by a confocal microscope (Zeiss) using a 40× objective. For T cells activity assay, the percentages of granzyme B+CD8⁺ T cells were determined by flow cytometry.

(8) Platelet Collagen Binding Assay.

Mouse collagen type I/III protein was purchased from Bio-Rad. The collagen solution (2.0 mg ml in 0.25% acetic acid) was coated on the confocal well overnight at 4° C. After that, the wells were blocked with 2% BSA before the binding assay. The EGFP-PD-L1 platelets (˜1×10⁸) were added in the collagen coated well for 30 s, then the wells were washed three times to remove the unbound platelets. Confocal microscopy (Zeiss) was used to observe the bind platelets using a 40× objective.

(9) Platelet Aggregation Assay.

Aggregation of platelets was assessed by confocal imaging. The platelets were labeled with WGA Alexa Fluor 594. Then the platelets were loaded to the confocal well and stimulated with 0.5 IU⁻¹ of thrombin for 30 min. Confocal microscopy was performed on a confocal microscope (Zeiss) in sequential scanning mode using a 63× objective.

(10) In Vivo Circulation Analysis.

The isolated platelets were labeled with NHS-Cy5.5. After that, the platelets were washed with PBS to remove the free NHS-Cy5.5. Then, the NOD mice were injected with the NHS-Cy5.5-labeled platelets (200 μL, ˜2×10⁸) through tail-vein. The blood of the NOD mice was collected after the platelet injection at different time points (at 2 min, 30 min, 1 h, 2 h, 4 h, 8 h, 24 h and 48 h, respectively). The serum was purified by centrifugation at 1500 rpm for 5 min, and the fluorescence of platelets was measured with TeCan Infinite M200 reader.

(11) In Vivo Biodistribution Analysis.

The isolated platelets were labeled with NHS-Cy5.5 in PBS buffer. Following incubation for 20 h, NHS-Cy5.5-labeled platelets were washed with PBS to remove the free NHS-Cy5.5 for three times. The NOD mice were injected with Cy5.5-labeled platelets (200 μL, ˜2×10⁸) through tail-vein. Then, the NOD mice were euthanized, and the major organs including pancreas, lung, heart, kidney, spleen, and liver were collected. Finally, the intensity of the major organs was recorded by a Xenogen IVIS Spectrum imaging system.

(12) Diabetic NOD Mice Treatment.

Female NOD/ShiLtJ mice were purchased from Jackson Lab (USA). All mouse studies were performed in the context of an animal protocol approved by the Institutional Animal Care and Use Committee at North Carolina State University and University of North Carolina at Chapel Hill. Overt diabetes was defined as blood glucose levels above 250 mg/dL for 2 consecutive days. Measurements were carried out by tail bleeding. The blood glucose of NOD mice was monitored starting at 10 weeks of age. Once the mouse on hyperglycemia (>250 mg/dL) for two days, the hyperglycemia mice were left untreated (control group) or injected with free platelets (˜2×10⁸) or PD-L1 platelets (˜2×10⁸) via the tail vein every 2 days. The blood glucose of NOD mice was measuring every two days up to a specific endpoint (40 days), and then the mice were sacrificed for further analysis.

(13) Tissue Immunofluorescent Assay.

The pancreases of the NOD mice were collected and frozen in optimal cutting medium (O.C.T.). The pancreas samples were cut using a cryotome and mounted on slides. The frozen pancreatic sections firstly were washed with PBS for 5 min to remove the O.C.T. Then, the specimens were blocked using the buffer containing 3% BSA and 0.2% Triton-X100. After that, the specimens were incubated with insulin, glucagon, and CD8 primary antibodies (1:100 in 1.5% BSA) overnight as indicated. The specimens were washed for three times with PBS for 5 min each. Subsequently, the specimens were incubated with FITC and TRITC labeled secondary antibody (diluted in 1.5% BSA) for 1 h. Finally, the nucleus of the samples was stained with DAPI for 20 min and was washed for three times with PBS. The samples were observed through the Confocal microscopy (Zeiss) using a 40× objective.

(14) Pancreas T Cell Analysis.

To evaluate the status of the pancreas infiltrated T cells, the pancreas was collected from the NOD mice with different treatments as indicated. The pancreas was dissociated to generate single-cell. The samples were passed through a 70-micron filter. Subsequently, the cells were stained with APC anti-mouse CD3 antibody, FITC-conjugated anti-CD4, PE-conjugated anti-CD8, PE-conjugated anti-FoxP3, FITC-conjugated anti-Granzyme B, and FITC-conjugated anti-IFN-γ as indicated. The percentages of CD3+CD8⁺ T cells, CD3CD4 T cells, granzyme B+CD8⁺ T cells, and IFN-γ+CD8⁺ T cells, and FoxP3+CD4⁺ Treg cells were determined by flow cytometry.

(15) Statistical Analysis.

All data were shown as the mean±s.d. Biological replicates were performed in all experiments unless otherwise stated. One-way or two-way analysis of variance (ANOVA) and Tukey post-hoc tests were used to analyze the samples with multiple comparisons. Survival data was analyzed using a log-rank test. All statistical analyses were carried out with the IBM SPSS statistics. p*<0.05 were considered statistically significant.

### E. REFERENCES

Akbarpour, M. et al. Insulin B chain 9-23 gene transfer to hepatocytes protects from type 1 diabetes by inducing Ag-specific FoxP3⁺ Tregs. Science translational medicine 7, 289ra281, doi:10.1126/scitranslmed.aaa3032 (2015).
Ali, R. A., Wuescher, L. M. & Worth, R. G. Platelets: essential components of the immune system. Current trends in immunology 16, 65-78 (2015).
Ansari, M. J. et al. The programmed death-1 (PD-1) pathway regulates autoimmune diabetes in nonobese diabetic (NOD) mice. The Journal of experimental medicine 198, 63-69, doi:10.1084/jem.20022125 (2003).
Anselmo, A. C. et al. Platelet-like nanoparticles: mimicking shape, flexibility, and surface biology of platelets to target vascular injuries. ACS nano 8, 11243-11253, doi:10.1021/nn503732m (2014).
Barclay, J., Creswell, J. & Leon, J. Cancer immunotherapy and the PD-1/PD-L1 checkpoint pathway. Archivos espanoles de urologia 71, 393-399 (2018).
Ben Nasr, M. et al. PD-L1 genetic overexpression or pharmacological restoration in hematopoietic stem and progenitor cells reverses autoimmune diabetes. Sci Transl Med 9, doi:10.1126/scitranslmed.aam7543 (2017).
Bluestone, J. A. et al. Type 1 diabetes immunotherapy using polyclonal regulatory T cells. Science translational medicine 7, 315ra189, doi:10.1126/scitranslmed.aad4134 (2015).
Bluestone, J. A., Herold, K. & Eisenbarth, G. Genetics, pathogenesis and clinical interventions in type 1 diabetes. Nature 464, 1293-1300 (2010).
Boldison, J. & Wong, F. S. Immune and Pancreatic beta Cell Interactions in Type 1 Diabetes. Trends in endocrinology and metabolism: TEM 27, 856-867, doi:10.1016/j.tem.2016.08.007 (2016).
Clemente-Casares, X. et al. Expanding antigen-specific regulatory networks to treat autoimmunity. Nature 530, 434-440, doi:10.1038/nature16962 (2016).
Gauci, M. L. et al. Autoimmune diabetes induced by PD-1 inhibitor-retrospective analysis and pathogenesis: a case report and literature review. Cancer immunology, immunotherapy: CH 66, 1399-1410, doi:10.1007/s00262-017-2033-8 (2017).
Hu, C. M. et al. Nanoparticle biointerfacing by platelet membrane cloaking. Nature 526, 118-121, doi:10.1038/nature15373 (2015).
Hu, Q. et al. Anticancer Platelet-Mimicking Nanovehicles. Adv Mater 27, 7043-7050, doi:10.1002/adma.201503323 (2015).
Hughes, J. et al. Precipitation of autoimmune diabetes with anti-PD-1 immunotherapy. Diabetes care 38, e55-57, doi:10.2337/dc14-2349 (2015).
Kapke, J., Shaheen, Z., Kilari, D., Knudson, P. & Wong, S. Immune Checkpoint Inhibitor-Associated Type 1 Diabetes Mellitus: Case Series, Review of the Literature, and Optimal Management. Case reports in oncology 10, 897-909, doi:10.1159/000480634 (2017).
Kapur, R. & Semple, J. W. Platelets as immune-sensing cells. Blood advances 1, 10-14, doi:10.1182/bloodadvances.2016000067 (2016).
Katsarou, A. et al. Type 1 diabetes mellitus. Nature reviews. Disease primers 3, 17016, doi:10.1038/nrdp.2017.16 (2017).
Keymeulen, B. et al. Insulin needs after CD3-antibody therapy in new-onset type 1 diabetes. The New England journal of medicine 352, 2598-2608, doi:10.1056/NEJMoa043980 (2005).
Lefrancais, E. et al. The lung is a site of platelet biogenesis and a reservoir for haematopoietic progenitors. Nature 544, 105-109, doi:10.1038/nature21706 (2017).
Lehuen, A., Diana, J., Zaccone, P. & Cooke, A. Immune cell crosstalk in type 1 diabetes. Nature reviews. Immunology 10, 501-513, doi:10.1038/nri2787 (2010).
Machlus, K. R. & Italiano, J. E., Jr. The incredible journey: From megakaryocyte development to platelet formation. The Journal of cell biology 201, 785-796, doi:10.1083/jcb.201304054 (2013).
Mause, S. F., von Hundelshausen, P., Zernecke, A., Koenen, R. R. & Weber, C. Platelet microparticles: a transcellular delivery system for RANTES promoting monocyte recruitment on endothelium. Arteriosclerosis, thrombosis, and vascular biology 25, 1512-1518, doi:10.1161/01.ATV.0000170133.43608.37 (2005).
Mellati, M. et al. Anti-PD-1 and Anti-PDL-1 Monoclonal Antibodies Causing Type 1 Diabetes. Diabetes care 38, e137-138, doi:10.2337/dc15-0889 (2015).
Moreau, T. et al. Large-scale production of megakaryocytes from human pluripotent stem cells by chemically defined forward programming. Nature communications 7, 11208, doi:10.1038/ncomms11208 (2016).
Morrell, C. N., Aggrey, A. A., Chapman, L. M. & Modjeski, K. L. Emerging roles for platelets as immune and inflammatory cells. Blood 123, 2759-2767, doi:10.1182/blood-2013-11-462432 (2014).
Ovalle, F. et al. Verapamil and beta cell function in adults with recent-onset type 1 diabetes. Nature medicine 24, 1108-1112, doi:10.1038/s41591-018-0089-4 (2018).
Piccin, A., Murphy, W. G. & Smith, O. P. Circulating microparticles: pathophysiology and clinical implications. Blood reviews 21, 157-171, doi:10.1016/j.blre.2006.09.001 (2007).
Rachidi, S. et al. Platelets subvert T cell immunity against cancer via GARP-TGFbeta axis. Science immunology 2, doi:10.1126/sciimmunol.aai7911 (2017).
Roep, B. O. et al. Plasmid-encoded proinsulin preserves C-peptide while specifically reducing proinsulin-specific CD8(+) T cells in type 1 diabetes. Science translational medicine 5, 191ra182, doi:10.1126/scitranslmed.3006103 (2013).
Rondina, M. T. & Garraud, O. Emerging evidence for platelets as immune and inflammatory effector cells. Frontiers in immunology 5, 653, doi:10.3389/fimmu.2014.00653 (2014).
Ruggeri, Z. M. & Mendolicchio, G. L. Adhesion mechanisms in platelet function. Circulation research 100, 1673-1685, doi:10.1161/01.RES.0000267878.97021.ab (2007).
Semple, J. W., Italiano, J. E., Jr. & Freedman, J. Platelets and the immune continuum. Nature reviews. Immunology 11, 264-274, doi:10.1038/nri2956 (2011).
Siljander, P. R. Platelet-derived microparticles—an updated perspective. Thrombosis research 127 Suppl 2, 530-33, doi:10.1016/50049-3848(10)70152-3 (2011).
Sun, C., Mezzadra, R. & Schumacher, T. N. Regulation and Function of the PD-L1 Checkpoint. Immunity 48, 434-452, doi:10.1016/j.immuni.2018.03.014 (2018).
Ueno, H. et al. Dendritic cell subsets in health and disease. Immunological reviews 219, 118-142, doi:10.1111/j.1600-065X.2007.00551.x (2007).
von Hundelshausen, P. & Weber, C. Platelets as immune cells: bridging inflammation and cardiovascular disease. Circulation research 100, 27-40, doi:10.1161/01.RES.0000252802.25497.b7 (2007).
Wan, X. et al. Pancreatic islets communicate with lymphoid tissues via exocytosis of insulin peptides. Nature 560, 107-111, doi:10.1038/s41586-018-0341-6 (2018).
Xing, Y. & Hogquist, K. A. T-cell tolerance: central and peripheral. Cold Spring Harbor perspectives in biology 4, doi:10.1101/cshperspect.a006957 (2012).
Zamora, C. et al. Binding of Platelets to Lymphocytes: A Potential Anti-Inflammatory Therapy in Rheumatoid Arthritis. J Immunol 198, 3099-3108, doi:10.4049/jimmunol.1601708 (2017).
Zhang, X. et al. Engineering PD-1-Presenting Platelets for Cancer Immunotherapy. Nano letters, doi:10.1021/acs.nanolett.8b02321 (2018).
Zhang, X. et al. The effect of autophagy inhibitors on drug delivery using biodegradable polymer nanoparticles in cancer treatment. Biomaterials 35, 1932-1943, doi:10.1016/j.biomaterials.2013.10.034 (2014).
Zhou, Q. & Melton, D. A. Pancreas regeneration. Nature 557, 351-358, doi:10.1038/s41586-018-0088-0 (2018).
Zhou, Q. Regenerative medicine: Interspecies pancreas transplants. Nature 542, 168-169, doi:10.1038/nature21490 (2017).
Zou, W., Wolchok, J. D. & Chen, L. PD-L1 (B7-H1) and PD-1 pathway blockade for cancer therapy: Mechanisms, response biomarkers, and combinations. Science translational medicine 8, 328rv324, doi:10.1126/scitranslmed.aad7118 (2016).

1. An engineered platelet comprising membrane bound exogenous PD-L1.

2. The engineered platelet of claim 1, further comprising membrane bound CD40L and/or toll-like receptors.

3. The engineered platelet of claim 1, a targeting moiety.

4. The engineered platelet of claim 3, wherein the targeting moiety is a peptide, polypeptide, polymer, small molecule, nucleic acid, antibody, or sugar.

5. The engineered platelet of claim 3, wherein the targeting moiety targets the bone marrow, liver, spleen, pancreas, prostate, bladder, heart, lung, brain, skin, kidneys, ovaries, testis, lymph nodes, small intestines, large intestines, or stomach.

6. A method of treating/reducing diabetes in a subject comprising administering to the subject the engineered platelets of claim 1.

7. The method of claim 6, further comprising administering to the subject β-islet cells.

8. The method of claim 7, wherein the engineered platelets are administered at least 1, 2, 3, 4, 5, 6, 7, 8, 9, 10, 11, 12, 13, 14, 15, 16, 17, 18, 19, 20, 21, 22, 23, 24, 30, 36, 42, 48 hours, 3, 4, 5, 6, 7, 8, 9, 10, 11, 12, 13, 14 days, 3, 4, 5, 6, 7, 8 weeks prior to the administration of the 0-islet cells.

9. A method of treating/reducing/preventing/inhibiting graft vs. host disease (GvHD) in a subject comprising administering to the subject the engineered platelets of claim 1.

10. A method of treating/reducing/preventing/inhibiting an autoinflammatory condition comprising administering to the subject the engineered platelets of claim 1.

11. The method of claim 10, wherein the autoinflammatory condition is selected from the group consisting of Achalasia, Acute disseminated encephalomyelitis, Acute motor axonal neuropathy, Addison's disease, Adiposis dolorosa, Adult Still's disease, Agammaglobulinemia, Alopecia areata, Alzheimer's disease, Amyloidosis, Ankylosing spondylitis, Anti-GBM/Anti-TBM nephritis, Antiphospholipid syndrome, Aplastic anemia, Autoimmune angioedema, Autoimmune dysautonomia, Autoimmune encephalomyelitis, Autoimmune enteropathy, Autoimmune hemolytic anemia, Autoimmune hepatitis, Autoimmune inner ear disease (AIED), Autoimmune myocarditis, Autoimmune oophoritis, Autoimmune orchitis, Autoimmune pancreatitis, Autoimmune polyendocrine syndrome, Autoimmune retinopathy, Autoimmune urticaria, Axonal & neuronal neuropathy (AMAN), Baló disease, Behcet's disease, Benign mucosal emphigoid, Bickerstaff s encephalitis, Bullous pemphigoid, Castleman disease (CD), Celiac disease, Chagas disease, Chronic fatigue syndrome, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diabetes mellitus type 1, Discoid lupus, Dressler's syndrome, Endometriosis, Enthesitis, Eosinophilic esophagitis (EoE), Eosinophilic fasciitis, Erythema nodosum, Essential mixed cryoglobulinemia, Evans syndrome, Felty syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Inflamatory Bowel Disease (IBD), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus nephritis, Lupus vasculitis, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjögren's syndrome, Sperm & testicular autoimmunity, Stiff person syndrome (SPS), Subacute bacterial endocarditis (SBE), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Urticaria, Urticarial vasculitis, Uveitis, Vasculitis, Vitiligo, Vogt-Koyanagi-Harada Disease, and Wegener's granulomatosis (or Granulomatosis with Polyangiitis (GPA)).

12. The method of claim 11, wherein the autoinflammatory condition is rheumatoid arthritis.

13. The method of treating diabetes of claim 6, wherein the engineered platelets is administered to the patient at least once every 12, 14, 16, 18, 20, 22, 24, 26, 28, 30, 32, 34, 36, 38, 40, 42, 44, 46, 48 hours, once every 3, 4, 5, 6, 7, 8, 9, 10, 11, 12, 13, 14, 15, 16, 17, 18, 19, 20, 21, 22, 23, 24, 25, 26, 27, 28, 29, 30, 31 days, once every 2, 3, 4, 5, 6, 7, 8, 9, 10, 11, or 12 months.

14. The method of treating diabetes of claim 6, wherein the engineered platelets is administered at least 1, 2, 3, 4, 5, 6, 7 times per week.

15. The method of treating/reducing/preventing/inhibiting graft vs. host disease (GvHD) of claim 9, wherein the engineered platelets is administered to the patient at least once every 12, 14, 16, 18, 20, 22, 24, 26, 28, 30, 32, 34, 36, 38, 40, 42, 44, 46, 48 hours, once every 3, 4, 5, 6, 7, 8, 9, 10, 11, 12, 13, 14, 15, 16, 17, 18, 19, 20, 21, 22, 23, 24, 25, 26, 27, 28, 29, 30, 31 days, once every 2, 3, 4, 5, 6, 7, 8, 9, 10, 11, or 12 months.

16. The method of treating/reducing/preventing/inhibiting an autoinflammatory condition of claim 10, wherein the engineered platelets is administered to the patient at least once every 12, 14, 16, 18, 20, 22, 24, 26, 28, 30, 32, 34, 36, 38, 40, 42, 44, 46, 48 hours, once every 3, 4, 5, 6, 7, 8, 9, 10, 11, 12, 13, 14, 15, 16, 17, 18, 19, 20, 21, 22, 23, 24, 25, 26, 27, 28, 29, 30, 31 days, once every 2, 3, 4, 5, 6, 7, 8, 9, 10, 11, or 12 months.

17. The method of treating/reducing/preventing/inhibiting graft vs. host disease (GvHD) of claim 9, wherein the engineered platelets is administered at least 1, 2, 3, 4, 5, 6, 7 times per week.

18. The method of treating/reducing/preventing/inhibiting an autoinflammatory condition of claim 10, wherein the engineered platelets is administered at least 1, 2, 3, 4, 5, 6, 7 times per week.
